(19) United States
(12) Patent Application Publication
(10) Pub. No.: US 2003/0186340 A1
(43) Pub. Date: Oct. 2, 2003

(54) Epitope tag recognized by a monoclonal antibody to Rickettsia typhi

(76) Inventor: Myong-Joon Hahn, Suwon (KR)

Correspondence Address: Squire, Sanders & Dempsey L.L.P., 14th Floor, 801 S. Figueroa Street, Los Angeles, CA, 90017-5554, US

(21) Appl. No.: 10/199,263

(22) Filed: Jul. 18, 2002

Related U.S. Application Data
(62) Division of application No. 09/551,045, filed on Apr. 17, 2000.

(51) Int. Cl. G01N 33/554; G01N 33/569

(52) U.S. Cl. 435/7.32

(57) ABSTRACT

The epitope recognized by a mouse monoclonal antibody (MAb) to the crystalline surface layer protein (SLP) of Rickettsia typhi, SRT10, is mapped to ten amino acid residues (SRTag, TFIGAIATDT). The oligonucleotide sequence covering the epitope recognized by SRT10 is inserted to a mammalian expression vector together with multiple cloning sites. When the SRTag is fused in frame to the coding region of protein gene and expressed in bacteria or mammalian cells, the MAb SRT10 can detect the tagged protein by immunoblotting, immunocytochemistry, and immunoprecipitation.

### BACKGROUND OF THE INVENTION

This invention relates to an epitope tag recognized by monoclonal antibody to the crystalline surface layer protein (SLP) to Rickettsia typhi and a method for detecting the tagged protein using immunoblotting, immunocytochemistry, and immunoprecipitation.

Epitope tagging and the antibody to the epitope have been widely used in cellular and molecular biology research. When no antibody to a particular protein is available, construction of a fusion gene containing a particular epitope (Tag) and subsequent detection of its product by the anti-Tag antibody are valuable alternatives for the characterization of that protein.

For example, epitope tagging has been applied to elucidate intracellular location, post-translational modification, affinity purification, and interactions with other proteins of the tagged protein. Further, the immunogenic and antigenic determinants of a synthetic peptide and the corresponding antigenic determinants in the parent protein have been elucidated [Evan et al., Mol. Cell. Biol., 12, pp3610-3616 (1985); Wilson et al., Cell, 37, pp767-778 (1984)].

Furthermore, for the purpose of simultaneous expression of several ectopic genes and distinguishing the gene products from endogenous proteins, several different Tags along with sensitive and specific antibodies to such Tags are required.

Previously, the inventor made mouse monoclonal antibodies to the crystalline surface layer protein (SLP) of Rickettsia typhi and cloned the gene (slpT) encoding this protein [Hahn et al., Gene, 133, pp129-133 (1993)]. In this study, the inventor determined the epitope recognized by one of our monoclonal antibodies (SRT10, IgG2a) to ten amino acid residues of SLP. By tagging this epitope to a putative chloride channel protein, NCC27/CLIC1, which is not well characterized [Tulk et al., Am. J. Physiol., 274, pp1140-9 (1998); Valenzuela et al., J. Biol. Chem., 272, pp12575-82 (1997)], the inventor examined the usefulness of this epitope tag and SRT10 as tools for the molecular and cellular biology research.

### SUMMARY OF THE INVENTION

The object of the invention provides an epitope recognized by a mouse monoclonal antibody (MAb) to the crystalline surface layer protein (SLP) of Rickettsia typhi, SRT10, which is mapped to ten amino acid residues (SRTag, Thr Phe Ile Gly Ala Ile Ala Thr Asp Thr [SEQ ID NO: 1]).

Another object of the invention provides an oligonucleotide sequence [SEQ ID NO: 2] covering the epitope recognized by SRT10, which is inserted to a mammalian expression vector together with multiple cloning sites.

Further object of the invention provides a monoclonal or polyclonal antibody to the SRTag as an epitope for any tagged protein.

When the SRTag is fused in frame to the coding region of any protein gene and expressed in bacteria or mammalian cells, the MAb SRT10 can detect the tagged protein by immunoblotting, immunocytochemistry, and immunoprecipitation.

We suggest that this specific recognition of the SRTag by SRT10 is generally applicable to the research of cellular and molecular biology requiring the expression and detection of fusion proteins.

### BRIEF DESCRIPTION OF THE DRAWINGS

FIG. 1. Construction of SRT-NCC27/CLIC1 plasmid. Oligonucleotides containing the coding region of SRTag and multiple cloning sites were inserted to pCMV6. cDNA of NCC27/CLIC1 was subcloned to the BamHI/SalI sites of the prepared expression plasmid.

FIG. 2. SRT10 recognizes the tagged NCC27/CLIC1 by immunoblotting. Untransfected cell lysate was probed with 1 g/ml of SRT10 (lane 1). SRT-NCC27/CLIC1 transfected cell lysates were probed with 0.1 (lane 2), 1 (lane 3), 10 (lane 4), 100 (lane 5), or 1000 ng/ml (lane 6) of SRT10. Molecular size standards are indicated on the left in kilodoltons (kDa).

FIG. 3. SRT10 precipitates the tagged NCC27/CLIC1 from mammalian cell lysates. In lane 1, 75 g of total cell lysate (50% of input) was loaded. 150 g of cell lysates were precipitated with control antibody (lane 2) or SRT10 (lane 3-7). Precipitated tagged protein and antibody complexes were washed with washing buffer containing 0.25 M (lane 2 and 3), 0.5 M (lane 4), 0.75 M (lane 5), 1 M (lane 6), or 1.5 M (lane 7) NaCl. Molecular size standards are indicated on the left in kilodoltons (kDa).

FIG. 4. SRT10 recognizes intracellular SRT-NCC27/CLIC1. HeLa (A and B) and C2C12 cells (C and D) were transfected with the expression plasmid of SRT-NCC27/CLIC1, stained with SRT10, and examined by confocal microscopy.

### DETAILED DESCRIPTION OF THE INVENTION

### Materials and Methods

Epitope Mapping and Expression Plasmid Construct

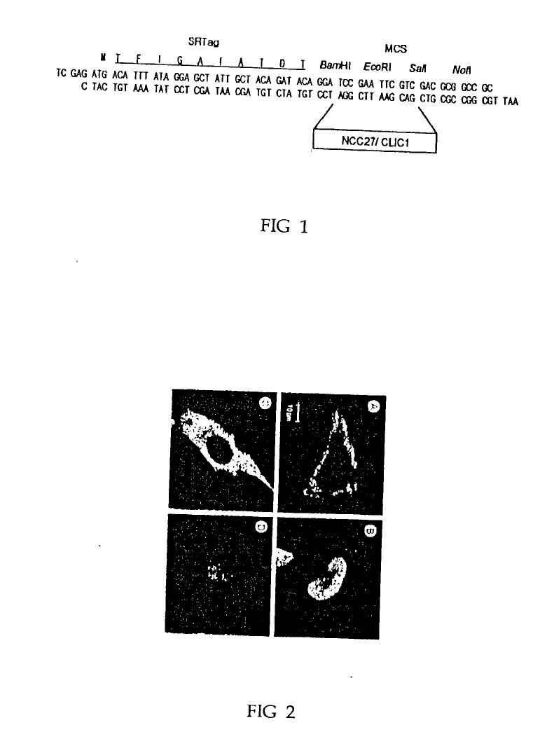

A series of deletion constructs of the slpT gene of R. typhi were prepared in pGEX™-4T-1 (Pharmacia, Uppsala, Sweden). By examining the reactivity of the SRT10 to bacterial lysates transformed with the expression constructs, we determined the epitope recognized by this MAb. On determining the epitope, its DNA sequence was inserted to the SalI/EcoRI sites of pCMV6 together with several cloning sites with oligonucleotides (FIG. 1). To this modified pCMV6, PCR-amplified cDNA of NCC27/CLIC1 (3, 4) was inserted. Oligonucleotides used for PCR were as follows: 5′ GACGGATCCATGGCTGAAGAACAAC [SEQ ID NO: 3]; and 5′ TCCCTCGAGGGGCTTATTTGAGGGC [SEQ ID NO: 4]. Underlining indicates the restriction sites (BamHI and XhoI, respectively). The resulting PCR product was cloned into the BamH1/Sal1 sites of the pGEX4T-1 and the tag-inserted pCMV6, for bacterial and mammalian cell expressions, respectively. Authenticity of the constructs was confirmed by automatic nucleotide sequencing.

Preparation of Rabbit Polysera to the NCC27/CLIC1

Recombinant GST-NCC27/CLIC1 fusion protein was prepared from E. coli. Purified GST-NCC27/CLIC1 was cleaved with biotinylated thrombin (Novagen, Madison, Wis., USA). Cleaved GST and thrombin was removed by glutathione Sepharose 4B (Pharmacia, Uppsala, Sweden) and streptavidin-agarose, respectively. The resulting cleaved and purified NCC27/CLIC1 was used as an immunogen injected into rabbits. Three injections were performed every two weeks. Two weeks after the last immunization, sera were collected.

Cell Culture and Transfection

Human embryonic kidney (HEK293), HeLa, and C₂C₁₂ cells were maintained in Dulbecco's modified Eagle's medium (DMEM) supplemented with 10% fetal calf serum. HEK293 cells were transiently transfected with the expression plasmid by a calcium phosphate precipitation method. Cells were incubated for 8 h with the transfection solution, washed with PBS, returned to culture with fresh media, and grown for a further 24 h. HeLa, and C₂C₁₂ cells were transiently transfected by Lipofectamine™ (Life Technologies, Gaithersburg, Md., USA) according to the manufacturer's instructions.

Immunoblot Analysis

Transfected HEK293 cells were lysed in 50 mM Tris (pH 8.0) and 0.5% NP40 containing protease inhibitors (1 mM phenylmethylsulfonyl fluoride, 2 mM aprotinin, and 2 mM leupeptin). Lysates were cleared by centrifugation at 15,000 g for 10 min. Protein contents of the resulting supernatants were determined by BCA™ kit (Pierce, Rockford, Ill., USA). Proteins (20 g per lane) were separated by SDS-PAGE using 10% polyacrylamide gels, transferred to nitrocellulose membrane, and probed with the monoclonal antibody, SRT10 (0.1, 1, 10, 100, or 1000 ng/ml). Untransfected cell lysate used as a control was probed with SRT10 at 1 g/ml. Sites of antibody binding were visualized by probing with alkaline phosphatase-conjugated goat anti-mouse IgG antibody (16 ng/ml, Santa Cruz Biotechnology, Santa Cruz, Calif., USA) followed by calorimetric detection with nitroblue tetrazolium and bromochloroindolyl phosphate.

Immuoprecipitation

Preparation of transfected cell lysates was performed with the same methods as above. Immuoprecipitation were performed by adding the SRT10 (4 g) to cell lysates (150 g) and incubating for an hour at 4° C. with constant rotation. Protein A-Sepharose beads (Pharmacia, Uppsala, Sweden) were then added, and incubated in the same condition for one more hour. Immune complexes were then centrifuged for 2 min at 4,000 g, washed twice in lysis buffer and twice in washing buffer (lysis buffer with 0.25, 0.5, 0.75, 1 or 1.5 M NaCl), and resuspended in SDS gel-loading buffer (50 mM Tris-HCl at pH 6.8, 100 mM dithiothreitol, 2% SDS, 0.1% bromophenol blue, 10% glycerol). Another monoclonal antibody to the SLP of R. typhi (IgG2a) was also used as a control for the precipitation and washed in washing buffer with 0.25 M NaCl. Samples were then analyzed by SDS-PAGE and immunoblotting. Blotted membrane was probed by the anti-NCC27/CLIC1 antibody described above. Alkaline phosphatase-conjugated goat anti-rabbit IgG antibody (0.4 g/ml, Santa Cruz biotechnology, Santa Cruz, Calif. USA) was used to detect antibody-binding sites.

Immunocytochiemistry

HeLa and C₂C₁₂ cells grown on microscope cover glasses and transfected with the expression plasmid of SRT-NCC27/CLIC1 were fixed with 4% paraformaldehyde in PBS and permeabilized with 0.1% Triton X-100. After washing in PBS, cells were incubated in SRT10 (3 g/ml), washed three times with PBS, incubated in FITC-conjugated goat anti-mouse igG antibody (30 g/ml, ICN Biochemicals, Aurora, Ohio, USA), washed three times with PBS, and mounted with FlouroGuard™ (BioRad, Hercules, Calif. USA). Prepared cells were examined by confocal microscopy (LSM 510, Carl Zeiss, Jena, Germany).

### Result and Discussion

Epitope Mapping

By the analysis of a series of deletion constructs of slpT gene of R. typhi, we determined the epitope recognized by the monoclonal antibody SRT10 as the ten amino acid residues, TFIGAIATDT (SRTag).

Immunoblot Analysis

To test whether the SRTag inserted in different sequence environment would affect its antigenicity, this epitope was fused in frame to the N-terminus of NCC27/CLIC1. This plasmid construct was transfected and the SRT-NCC27/CLIC1 was expressed in HEK293 cells. On measuring the protein content, 20 g of total cell lysate was subjected to immunoblotting. As shown in FIG. 2, SRT10 recognized the SRT-NCC27/CLIC1 even at 1 ng/ml of antibody concentration. Detectable cross-reactivity with the HEK293 proteins was not observed. When the antibody-binding site was probed with the goat anti-mouse IgG antibody at 80 ng/ml, this secondary antibody cross-reacted with several cellular proteins. However, at this concentration, signals from the tagged proteins were recognized at 0.1 ng/ml of SRT10 (data not shown). When we expressed the deletion constructs of SLP in E. coli and examined their products with immunoblotting, no detectable cross-reactivity with E. coli proteins was observed (data not shown). Thus this MAb can recognize the denatured tagged protein sensitively and specifically by immunoblotting.

Immunoprecipitation

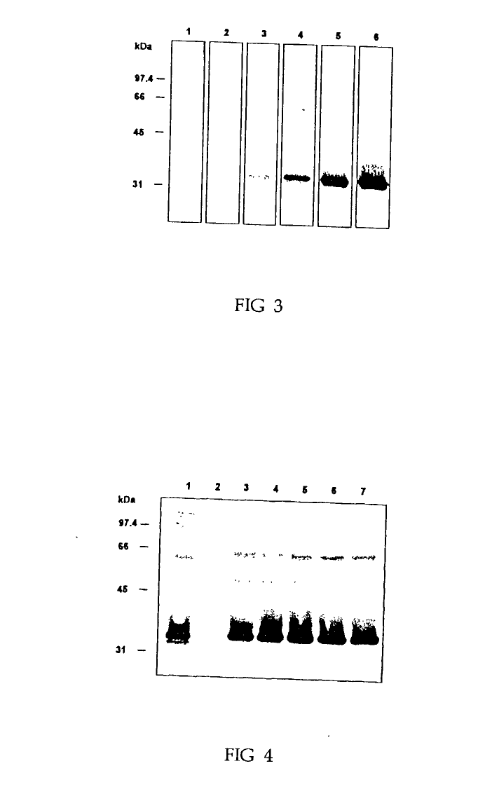

To test whether SRT10 could precipitate tagged protein from mammalian cell lysate, transfected 293HEK cells were subjected to immunoprecipitation (FIG. 3). Compared with the signal by the SRT-NCC27/CLIC1 from total cell lysate (FIG. 3, lane 1), about 70% of tagged proteins were precipitated by 4 g of SRT10 in this condition. When the concentrations of NaCl in washing buffers were increased from 0.25 M to 1.5 M, the amount of precipitated SRT-NCC27/CLIC1 did not decrease suggesting a high affinity binding of the SRT10 to the SRT-tagged NCC27/CLIC1. When the cell lysate was precipitated with a control antibody, precipitation of the tagged protein was not observed (FIG. 3, lane 2). Thus this antibody can precipitate the tagged protein from mammalian cell lysate efficiently.

Immunocytochemistry

To test whether SRT10 could detect intracellular tagged protein, transfected HeLa and C₂C₁₂ cells were subjected to immunocytochemistry and examined by confocal microscopy (FIG. 4). Most of the C₂C₁₂ cells were stained prominently in the cytoplasm; however, a few cells were stained prominently in the nucleus. In HeLa cells, the antibody stained dominantly the nucleus, however, some cells were stained dominantly in the cytoplasm. It is likely that during the cell cycle, the localization of the NCC27/CLIC1 may be changed between the nucleus and cytoplasm. In adjacent, nontransfected cells, no detectable staining was observed (data not shown). Thus this monoclonal antibody can detect the tagged protein by immunocytochemistry.

In this report, we determined the linear epitope recognized by the mouse monoclonal antibody (SRT10) to the rickettsial protein, SLP. By tagging this epitope to NCC27/CLIC1, we showed that SRT10 could detect this tagged protein by immunoblotting, immunoprecipitation, or immunocytochemistry. As this monoclonal antibody is both sensitive and specific, this tag and its monoclonal antibody should be generally applicable for cellular and molecular research.

### REFERENCES

1. Evan, G. I., Lewis, G. K., Ramsay, G., and Bishop, J. M. 1985. Isolation of monoclonal antibodies specific for human c-myc proto-oncogene product. Mol. Cell. Biol. 12:36103616.

2. Hahn, M. J., Kim, K. K., Kim, I., and Chang, W. H. 1993. Cloning and sequence analysis of the gene encoding the crystalline surface layer protein of Rickettsia typhi. Gene 133:129-133.

3. Tulk, B. M., Edwards, J. C. 1998. NCC27, a homolog of intracellular Cl-channel p64, is expressed in brush border of renal proximal tubule. Am. J. Physiol. 274:F1140-9

4. Valenzuela, S. M., Martin, D. K., Por, S. B., Robbins, J. M., Warton, K., Bootcov, M. R., Schofield, P. R., Campbell, T. J., and Breit, S. N. 1997. Molecular cloning and expression of a chloride ion channel of cell nuclei. J. Biol. Chem. 272:12575-82.

5. Wilson, I. A., Niman, H. L., Houghten, R. A., Cherenson, A. R., Connolly, M. L., and Lerner, R. A. 1984. The structure of an antigenic determinant in a protein. Cell 37:767-778.

What is claimed is:

1. An epitope recognized by a mouse monoclonal antibody (MAb), SRT10, to the crystalline surface layer protein (SLP) of Rickettsia typhi comprising SRTag consisting of ten amino acid sequence [SEQ ID NO: 1].

2. The epitope recognized by a mouse monoclonal antibody (MAb) according to claim 1, wherein an oligonucleotide sequence [SEQ ID NO: 2] corresponding to SRTag is inserted to an expression vector.

3. The epitope recognized by a mouse monoclonal antibody (MAb) according to claim 1, wherein the tagged protein is detected by MAb (SRT10) when the oligonucleotide sequence [SEQ ID NO: 2] corresponding to SRTag is fused to any protein gene desired to be expressed and detected in bacteria or mammalian cells.

4. Monoclonal or polyclonal antibody to the SRTag as an epitope for any tagged protein.

5. The application of epitope of claim 1 for the research of cellular and molecular biology requiring the expression and detection of fusion proteins.


SEQUENCE LISTING (4 sequences)

SEQ ID NO: 1 - protein, 10 aa, Rickettsia typhi
TFIGAIATDT

SEQ ID NO: 2 - DNA, 30 bp, Rickettsia typhi
acatttataggagctattgctacagataca

SEQ ID NO: 3 - DNA, 25 bp, Artificial Sequence
  PCR primer
gacggatccatggctgaagaacaac

SEQ ID NO: 4 - DNA, 25 bp, Artificial Sequence
  PCR primer
tccctcgaggggcttatttgagggc